(19) United States
(12) Patent Application Publication
(10) Pub. No.: US 2018/0158180 A1
(43) Pub. Date: Jun. 7, 2018

(54) IMAGE PROCESSING APPARATUS

(71) Applicant: HOYA CORPORATION, Tokyo (JP)

(72) Inventor: Fumika YOKOUCHI, Tokyo (JP)

(73) Assignee: HOYA CORPORATION, Tokyo (JP)

(21) Appl. No.: 15/308,479

(22) PCT Filed: Mar. 15, 2016
(86) PCT No.: PCT/JP2016/058112

§ 371 Date: Nov. 2, 2016

(30) Foreign Application Priority Data
Apr. 1, 2015 (JP) 2015-074763

(51) Int. Cl. G06T 5/00; H04N 9/64; H04N 9/78; A61B 1/313

(52) U.S. Cl. CPC G06T 5/009; H04N 9/646; H04N 9/045; A61B 1/3137; H04N 9/78

(57) ABSTRACT

An image processing apparatus includes an image data acquiring means for acquiring image data that expresses a captured image of biological tissue, and a YC separation processing means for performing signal processing in which a luminance signal and color signals are generated based on the RGB signal of the image data. The ratio of the R component of the RGB signal included in the luminance signal is greater than the ratio of the G component and the ratio of the B component.

### TECHNICAL FIELD

The present invention relates to an image processing apparatus that performs image processing on an endoscopic image.

### BACKGROUND ART

An endoscope apparatus is known that captures an endoscopic image that includes a high-contrast image of deep blood vessels (referred to hereinafter as an “enhanced deep blood vessel image”) by using illumination light with a narrow band whose peak is in the absorption wavelength region of hemoglobin (referred to hereinafter as “special light”). Japanese Patent No. 5362149 discloses an example of this type of endoscope apparatus.

### SUMMARY OF INVENTION

Conventionally, using special light when capturing an enhanced deep blood vessel image has required endoscopic observation to be performed using a special light source apparatus that is equipped with a narrowband optical bandpass filter and a narrowband light source for producing special light separately from a white light source for normal observation.

The present invention was achieved in light of the aforementioned situation, and an object thereof is to provide an image processing apparatus that can generate an enhanced deep blood vessel image without using a special light source apparatus.

An image processing apparatus according to one embodiment of the present invention includes: an image data acquiring means for acquiring image data expressing a captured image of biological tissue; and a YC separation processing means for performing signal processing in which a luminance signal and a color signal are generated based on an RGB signal of the image data, wherein a ratio of an R component of the RGB signal included in the luminance signal is greater than a ratio of a G component and a ratio of a B component.

In the above-described image processing apparatus, the signal processing may include standard signal processing in which the image does not change substantially before and after the signal processing, and special signal processing in which the luminance signal that is output includes a larger amount of the R component than both the G component and the B component, the image processing apparatus may include: a selecting means for selecting whether the standard signal processing or the special signal processing is to be performed, and the YC separation processing means may perform the signal processing that was selected by the selecting means.

In the above-described image processing apparatus, the YC separation processing means may perform the signal processing with use of a matrix operation that uses a color matrix, and the YC separation processing means may use a standard color matrix in the standard signal processing, and use a special color matrix in the special signal processing.

In the above-described image processing apparatus, the YC separation processing means may include: a memory that stores the standard color matrix and the special color matrix; a matrix selection unit that selects one of the standard color matrix and the special color matrix and reads out the selected matrix from the memory; and a computation unit that performs the matrix operation with use of the matrix that was read out by the matrix selection unit.

In the above-described image processing apparatus, the luminance signal may be proportional to the R component of the RGB signal.

In the above-described image processing apparatus, the luminance signal may include an element obtained by multiplying the R component of the RGB signal by a gain constant, and the image processing apparatus may include: a means for changing the gain constant.

The above-described image processing apparatus may include: an automatic gain adjusting means for automatically adjusting the gain constant based on the luminance signal.

In the above-described image processing apparatus, the ratio of the R component of the RGB signal included in the luminance signal may be greater than a sum of the ratio of the C component and the ratio of the B component.

In the above-described image processing apparatus, the ratio of the R component of the RGB signal included in the luminance signal may be greater than or equal to 50%.

In the above-described image processing apparatus, the color signal may be made up of two color difference signals.

In the above-described image processing apparatus, the YC separation processing means may generate a YCrCb signal, a YPrPb, or a YUV signal.

An image processing apparatus according to an embodiment of the present invention includes: an image data acquiring means for acquiring image data expressing a captured image of biological tissue; and a YC separation processing means for performing signal processing in which a luminance signal and a color signal are generated based on an RGB signal of the image data, wherein the signal processing includes standard signal processing in which the image does not change substantially before and after the signal processing, and special signal processing in which the luminance signal that is output includes a larger amount of the R component of the RGB signal than in the standard signal processing, the image processing apparatus includes: a selecting means for selecting whether the standard signal processing or the special signal processing is to be performed, and the YC separation processing means performs the signal processing that was selected by the selecting means.

According to an embodiment of the present invention, it is possible to obtain an enhanced deep blood vessel image without using a special light source apparatus.

### BRIEF DESCRIPTION OF DRAWINGS

FIG. 1 is a block diagram showing a configuration of an electronic endoscope system according to an embodiment of the present invention.

FIG. 2 is a block diagram showing a configuration of a pre-stage signal processing circuit according to the embodiment of the present invention.

FIG. 3 is a diagram illustrating image information of various color components of a normal observed image.

### DESCRIPTION OF EMBODIMENTS

Hereinafter an embodiment of the present invention will be described with reference to the drawings. Note that an electronic endoscope system is taken as an example of one embodiment of the present invention in the following description.

Overall Configuration of Electronic Endoscope System 1

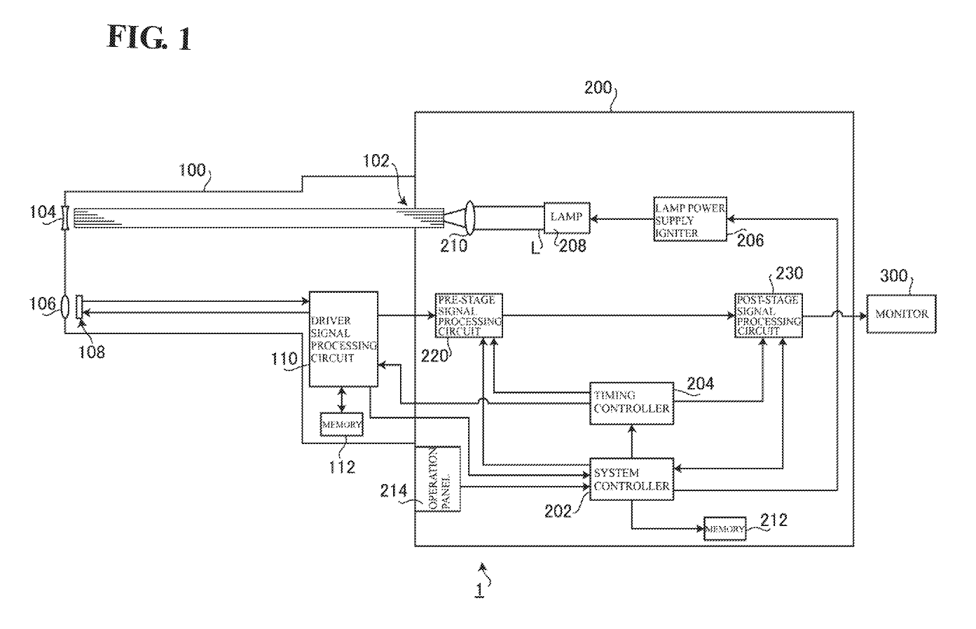

FIG. 1 is a block diagram showing the schematic configuration of an electronic endoscope system 1 of the present embodiment. As shown in FIG. 1, the electronic endoscope system 1 includes an electronic endoscope 100, a processor 200, and a monitor 300.

The processor 200 includes a system controller 202 and a timing controller 204. The system controller 202 executes various programs stored in a memory 212 and performs overall control of the electronic endoscope system 1. Also, the system controller 202 is connected to an operation panel 214. The system controller 202 changes operations of the electronic endoscope system 1 and operation parameters in accordance with instructions from an operator that are input using the operation panel 214. The timing controller 204 outputs a clock pulse, which is for adjustment of the timing of the operations of portions, to circuits in the electronic endoscope system 1.

A lamp 208 is activated by a lamp power supply igniter 206, and thereafter emits irradiation light L. The lamp 208 is an LED (Light Emitting Diode) or a high-intensity lamp such as a xenon lamp, a halogen lamp, a mercury lamp, or a metal halide lamp. The irradiation light L is light having a spectrum that mainly spreads from the visible light region to the invisible infrared light region (or is white light that includes at least the visible light region).

The irradiation light L emitted by the lamp 208 is condensed onto the entrance end face of an LCB (Light Carrying Bundle) 102 by a condensing lens 210, and enters the LCB 102.

The irradiation light L that entered the LCB 102 propagates inside the LCB 102 and exits from the exit end face of the LCB 102 arranged at the tip of the electronic endoscope 100, and then passes through a light distribution lens 104 and irradiates a subject. Returning light from the subject that was irradiated with the irradiation light L passes through an objective lens 106 and is formed into an optical image on the light receiving surface of a solid-state imaging element 108.

The solid-state imaging element 108 is a single-plate color CCD (Charge Coupled Device) image sensor that has a Bayer pixel arrangement. The solid-state imaging element 108 accumulates charge according to the light quantity of an optical image formed on pixels on the light receiving surface, generates R (Red), G (Green), and B (Blue) imaging signals, and outputs the imaging signals. Note that the solid-state imaging element 108 is not limited to being a CCD image sensor, and a CMOS (Complementary Metal Oxide Semiconductor) image sensor or another type of imaging apparatus may be employed. The solid-state imaging element 108 may be an element that includes a complementary color filter.

A driver signal processing circuit 110 is provided in the connection portion of the electronic endoscope 100. The driver signal processing circuit 110 receives imaging signals from the solid-state imaging element 108 at a field cycle. Note that the terms “field” and “frame” may be switched in the following description. In the present embodiment, the field cycle and the frame cycle are respectively 1/60 seconds and 1/30 seconds. The driver signal processing circuit 110 performs predetermined processing on the imaging signals received from the solid-state imaging element 108, and outputs the resulting signals to a pre-stage signal processing circuit 220 of the processor 200.

The driver signal processing circuit 110 accesses the memory 112 and reads out unique information regarding the electronic endoscope 100. The unique information regarding the electronic endoscope 100 recorded in the memory 112 includes, for example, the pixel count, sensitivity, operable field rate, and model number of the solid-state imaging element 108. The unique information read out from the memory 112 is output by the driver signal processing circuit 110 to the system controller 202.

The system controller 202 generates a control signal by performing various computation based on the unique information regarding the electronic endoscope 100. The system controller 202 uses the generated control signal to control the operations of and the timing of various circuits in the processor 200 so as to perform processing suited to the electronic endoscope that is connected to the processor 200.

The timing controller 204 supplies a clock pulse to the driver signal processing circuit 110 in accordance with timing control performed by the system controller 202. In accordance with the clock pulse supplied from the timing controller 204, the driver signal processing circuit 110 controls the driving of the solid-state imaging element 108 according to a timing synchronized with the field rate of the images processed by the processor 200.

The pre-stage signal processing circuit 220 performs predetermined signal processing such as color interpolation, a matrix operation, and Y/C separation on the image signals received from the driver signal processing circuit 110 in one field cycle, and outputs the resulting signals to a post-stage signal processing circuit 230. Details regarding the pre-stage signal processing circuit 220 will be described later.

The post-stage signal processing circuit 230 processes the image signals received from the pre-stage signal processing circuit 220 to generate screen data for monitor display, and converts the generated screen data for monitor display into a video signal in a predetermined video format. The resulting video signal is output to a monitor 300. Accordingly, color images of the subject are displayed on the display screen of the monitor 300.

Configuration of Pre-Stage Signal Processing Circuit 220

The processor 200 of the present embodiment operates in two operating modes. One is a normal display mode in which normal observed images N are displayed on the screen of the monitor 300, and the other is an enhanced deep blood vessel display mode in which enhanced deep blood vessel images E obtained by deep blood vessel enhancement processing are displayed on the screen of the monitor 300. These two operating modes are realized by a YC separation processing unit 228 of the pre-stage signal processing circuit 220 that will be described below.

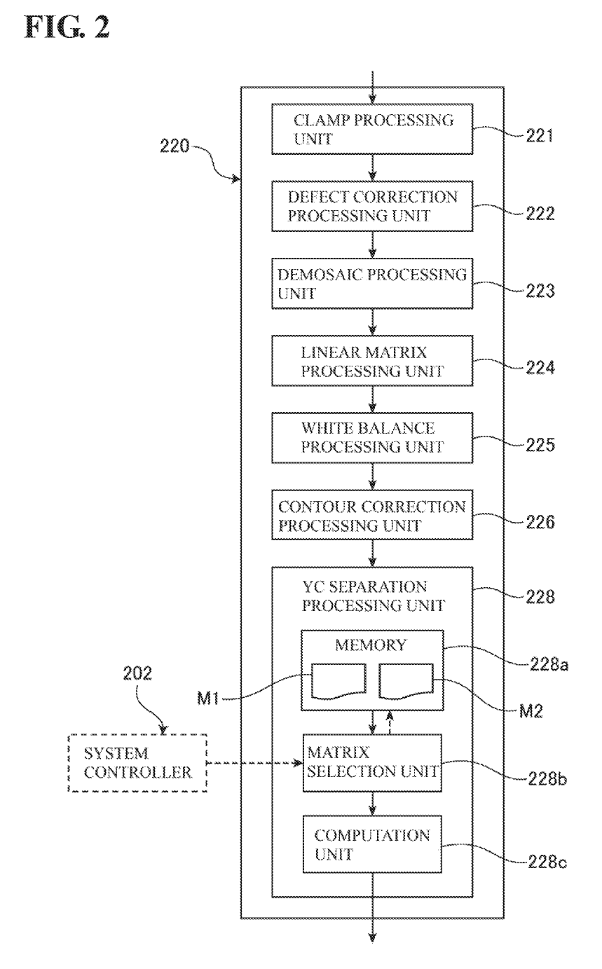

FIG. 2 is a block diagram showing the configuration of the pre-stage signal processing circuit 220 of the present embodiment. The pre-stage signal processing circuit 220 includes a clamp processing unit 221, a defect correction processing unit 222, a demosaic processing unit 223, a linear matrix processing unit 224, a white balance processing unit 225, a contour correction processing unit 226, and the YC separation processing unit 228.

The clamp processing unit 221 is a function block that performs clamp processing for removing an offset component from an image signal.

The defect correction processing unit 222 is a function block that performs defect correction processing for correcting the pixel values of defect pixels using the pixel values of surrounding pixels.

The demosaic processing unit 223 is a function block that performs demosaic processing (interpolation processing) in which captured image data (RAW data) that is made up of pixels having single-color color information is converted into image data that is made up of pixels having full-color pixel values.

The linear matrix processing unit 224 is a function block that performs linear matrix processing for correcting the spectral characteristics of the imaging element using a color matrix.

The white balance processing unit 225 is a function block that performs white balance processing for correcting the spectral characteristics of the illumination light.

The contour correction processing unit 226 is a function block that performs contour correction processing for compensating for degradation in the spatial frequency characteristics of the image signal.

Configuration of YC Separation Processing Unit 228

The YC separation processing unit 228 is a function block that performs YC separation processing for converting an RGB signal into a luminance signal Y and color signals C (color difference signals Cb and Cr) using a matrix circuit.

The YC separation processing unit 228 of the present embodiment can switch between executing two types of YC separation processing, namely standard YC separation processing (standard signal processing) and special YC separation processing (special signal processing) that pertains to this embodiment of the present invention.

Standard YC separation processing is normal YC separation processing that is executed in the normal display mode, and in this processing, only color space conversion is performed on the RGB signal of a normal observed image N output from the contour correction processing unit 226, and a YCrCb signal (luminance/color difference signal) for a normal observed image N is output. The image does not change substantially due to this standard YC separation processing.

Special YC separation processing is performed in the enhanced deep blood vessel display mode, and in this processing, a normal observed image N is subjected to color balance adjustment for enhancing deep blood vessels without changing the image tint during color space conversion, thus generating a YCrCb signal for an enhanced deep blood vessel image E. The luminance signal output in this special YC separation processing includes a larger amount of the R component of the RGB signal than in the standard YC separation processing.

As shown in FIG. 2, the YC separation processing unit 228 includes a memory 228a, a matrix selection unit 228b, and a computation unit 228c.

The memory 228a stores two types of color matrices (standard color matrix M1 and special color matrix M2).

The matrix selection unit 228b selects the color matrix that is to be used under control of the system controller 202, reads out the selected color matrix from the memory 228a, and supplies it to the computation unit 228c.

The computation unit 228c performs standard YC separation processing or special YC separation processing using the color matrix supplied from the matrix selection unit 228b.

The standard color matrix M1 is a normal color matrix that is used in standard YC separation processing, and is compliant with the ITU-R BT.601 standard.

Expression 1 below is a transformation expressing signal conversion that is performed with use of the standard color matrix M1 in standard YC separation processing.

$$[ \begin{matrix}Y \\ Cr \\ Cb\end{matrix} ] = M 1 [ \begin{matrix}R \\ G \\ B\end{matrix} ] = [ \begin{matrix}0.299 & 0.587 & 0.114 \\ - 0.169 & - 0.331 & 0.500 \\ 0.500 & - 0.419 & - 0.081\end{matrix} ] [ \begin{matrix}R \\ G \\ B\end{matrix} ] = [ \begin{matrix}0.299 R + 0.587 G + 0.114 B \\ - 0.169 R - 0.331 G + 0.500 B \\ 0.500 R - 0.0419 G - 0.081 B\end{matrix} ] \qquad Expression 1$$

The RGB signal color components in the luminance signal Y generated by standard YC separation processing (Expression 1) are mixed in a ratio that corresponds to the standard luminosity function. For this reason, the luminance signal Y includes a large green (G) component, and only a very small red (R) component. The weighting of these color components produces an image that appears to have the same brightness as before standard YC separation processing.

The special color matrix M2 is a special-purpose color matrix that is used in special YC separation processing.

Expression 2 below is a transformation expressing signal conversion that is performed with use of the special color matrix M2 in special YC separation processing.

$$[ \begin{matrix}Y \\ Cr \\ Cb\end{matrix} ] = M 2 [ \begin{matrix}R \\ G \\ B\end{matrix} ] = [ \begin{matrix}k & 0.000 & 0.000 \\ - 0.169 & - 0.331 & 0.500 \\ 0.500 & - 0.419 & - 0.081\end{matrix} ] [ \begin{matrix}R \\ G \\ B\end{matrix} ] = [ \begin{matrix}kR \\ - 0.169 R - 0.331 G + 0.500 B \\ 0.500 R - 0.0419 G - 0.081 B\end{matrix} ] \qquad Expression 2$$

Note that the gain constant k is a positive number less than or equal to 1.

In the YCrCb signal for an enhanced deep blood vessel image E that is generated in special YC separation processing, the color difference signals Cr and Cb have the same values as in a normal observed image N generated in standard YC separation processing, but the YCrCb signal for an enhanced deep blood vessel image E generated in special YC separation processing is different from the YCrCb signal for a normal observed image N generated in standard YC separation processing in that the luminance signal is made up of only the red (R) component.

Illumination light that is emitted onto biological tissue penetrates to a certain depth in the biological tissue while also being scattered by the biological tissue, and a portion of the illumination light forms an image on the light receiving surface of the solid-state imaging element 108. The shorter the wavelength of the light is, the more it is scattered by the biological tissue and therefore cannot penetrate deeply into the biological tissue. Conversely, the longer the wavelength of the light is, the less it is scattered and the more it can penetrate relatively deeply into the biological tissue. Also, blood (hemoglobin) absorbs almost no light in the wavelength region of 600 nm or more, and therefore red light can penetrate more deeply into biological tissue than blue light or green light can, and an optical image of blood vessels that include a large amount of blood can be formed more vividly.

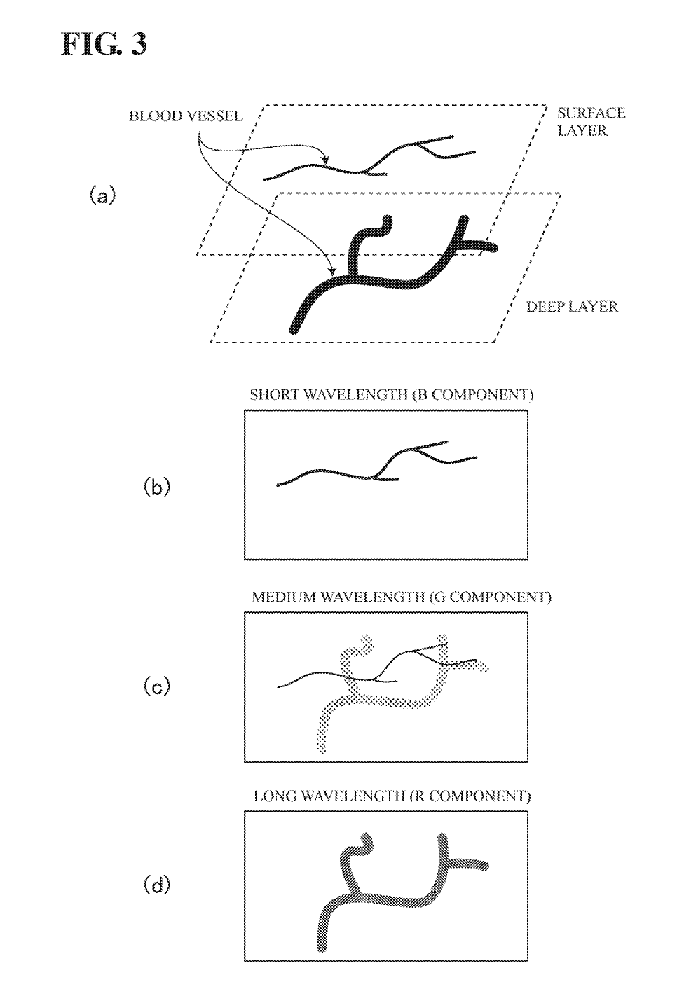

As a result, as shown in FIG. 3, the red (R) component of the endoscopic image includes a large amount of information regarding deep blood vessels (FIG. 3(d)), whereas the blue (B) component includes a large amount of information regarding the surface layer portion of the biological tissue (FIG. 3(b)). Also, the green (G) component includes information regarding both deep portions and the surface layer portion of the biological tissue (FIG. 3(c)).

In an enhanced deep blood vessel image E generated by the special YC separation processing of the present embodiment, the luminance Y is determined by intensity of the red (R) component (specifically, proportionally to the red (R) component), and therefore this image includes a large amount of information regarding deep blood vessels and a small amount of information regarding the surface (i.e., deep blood vessels are enhanced). Also, the color difference signals that determine the tint of the image have the same values as the normal observed image N, and thus an image in which deep blood vessels are enhanced while maintaining a natural tint is obtained.

Operations of YC Separation Processing Unit 228

Next, operations of the YC separation processing unit 228 will be described. The normal display mode and the enhanced deep blood vessel display mode are switched by a user input operation performed on the operation panel 214. When a user input operation for selecting the enhanced deep blood vessel display mode is performed, an instruction for switching to the enhanced deep blood vessel display mode is output from the system controller 202 to the YC separation processing unit 228. The matrix selection unit 228b receives the instruction for switching to the enhanced deep blood vessel display mode, reads out the special color matrix M2 from the memory 228a, and transmits it to the computation unit 228c. The computation unit 228c then performs special YC separation processing on the RGB signal of a normal observed image N output from the contour correction processing unit 226 based on the special color matrix M2 that was last received from the matrix selection unit 228b, thus generating a YCbCr signal for an enhanced deep blood vessel image E.

Also, when a user input operation for selecting the normal display mode is performed on the operation panel 214, an instruction for switching to the normal display mode is output from the system controller 202 to the YC separation processing unit 228. The matrix selection unit 228b receives the instruction for switching to the normal display mode, reads out the standard color matrix M1 from the memory 228a, and transmits it to the computation unit 228c. The computation unit 228c then performs standard YC separation processing on the RGB signal of a normal observed image N output from the contour correction processing unit 226 based on the standard color matrix M1 that was last received from the matrix selection unit 228b, thus generating a YCbCr signal for a normal observed image N.

The YCbCr signal for an enhanced deep blood vessel image E (or a normal observed image N) generated by the YC separation processing unit 228 is converted into a video signal by the post-stage signal processing circuit 230 and output to the monitor 300, and then the enhanced deep blood vessel image E (or normal observed image N) is displayed on the display screen of the monitor 300.

Also, the gain value k of the special color matrix M2 is a changeable parameter, and the initial value thereof is set to the maximum value of 1.0. An endoscopic image has an intense red color component, and therefore there are cases where when the initial value is used, the luminance is saturated (or nearly saturated), and the contrast of the enhanced deep blood vessel image E decreases. For this reason, the gain value k can be changed by a user input operation performed on the operation panel 214.

When a user input operation for changing the setting of the gain value k is performed on the operation panel 214, an instruction for updating the gain value k to the value inputted by the user is output from the system controller 202 to the YC separation processing unit 228. The matrix selection unit 228b receives the gain value k update instruction, and rewrites the gain value k of the special color matrix M2 stored in the memory 228a to the value inputted by the user. The luminance of the enhanced deep blood vessel image E is thus adjusted. Note that a configuration is possible in which the YC separation processing unit 228 automatically adjusts the gain value k based on the luminance distribution of the enhanced deep blood vessel image E, for example.

Variations

Next, a variation of special YC separation processing (special color matrix M2) will be described.

Expression 3 is a variation of the transformation in special YC separation processing (special color matrix M2) according to this embodiment of the present invention.

$$[ \begin{matrix}Y \\ Cr \\ Cb\end{matrix} ] = M 2 [ \begin{matrix}R \\ G \\ B\end{matrix} ] = [ \begin{matrix}0.600 k & 0.200 k & 0.200 k \\ - 0.169 & - 0.331 & 0.500 \\ 0.500 & - 0.419 & - 0.081\end{matrix} ] [ \begin{matrix}R \\ G \\ B\end{matrix} ] = [ \begin{matrix}k ( 0.600 R + 0.200 G + 0.200 B ) \\ - 0.169 R - 0.331 G + 0.500 B \\ 0.500 R - 0.0419 G - 0.081 B\end{matrix} ] \qquad Expression 3$$

Note that the gain constant k is a positive number less than or equal to 1.

In this variation, the luminance signal Y for an enhanced deep blood vessel image E includes the (G component and the B component of a normal observed image N as well. In this way, even when the luminance signal Y includes color components other than the R component, the effects of the present invention can be obtained if the R component that has the least information regarding the surface layer portion of the biological tissue (the ratio of the R signal included in the luminance signal Y, which is the coefficient “0.600” in Expression 3) is given the greatest weight.

If the weight of the R component is set to 2 times (more effectively 3 times, or even more effectively 5 times) or more the weight of the B component, a strong effect of enhancing deep blood vessels is obtained.

Also, if the weight of the R component is set to a weight that is 1.20 times or more the weight of the G component (more effectively a weight that is 2 times the weight of the G component, even more effectively a weight that is 3 times the weight of the G component, or further more effectively a weight that is 5 times the weight of the G component), an even stronger effect of enhancing deep blood vessels is obtained.

Also, if the weight of the R component is set higher than the sum of the weights of the G component and the B component, an image in which the deep blood vessels are even more enhanced is obtained.

Moreover, if the weight of the R component is set to a value greater than or equal to 2 times the sum of the weights of the G component and the B component (more effectively 3 times the sum of the weights of the G component and the B component, or further more effectively 5 times the sum of the weights of the G component and the B component), an image in which the deep blood vessels are even more enhanced is obtained.

Also, if the weight of the R component is set to 0.5 (50% the total sum of the weights of the color components) or more, an image in which the deep blood vessels are even more enhanced is obtained.

The foregoing description is a description of an illustrative embodiment of the present invention. The embodiments of the present invention are not limited to the foregoing description, and various modifications can be made within the scope of the technical idea of the present invention. For example, the embodiments of the present invention also include appropriate combinations of embodiments and the like explicitly specified in this specification and obvious embodiments and the like.

For example, in the example in the above embodiment, the present invention is applied to an apparatus that generates a YCbCr signal, but the present invention can also be applied to an apparatus that generates another type of luminance/color difference signal (e.g., a YLUV signal or a YPbPr signal).

Also, the processor 200 (image processing apparatus) of the above embodiment is configured to operate in two modes, namely the normal display mode for displaying normal observed images N on the monitor and the enhanced deep blood vessel display mode for displaying enhanced deep blood vessel images E on the monitor, but it may be configured to operate in three or more operating modes, including an operating mode for generating screen data in which normal observed images N and enhanced deep blood vessel images E are displayed side-by-side in one screen by image compositing, and displaying this screen data on the monitor (twin mode).

Also, in the configuration of the above embodiment, the operating mode is switched by a user input operation performed on the operation panel 214, but a configuration is possible in which, for example, a mode switching button is provided on the control body of the electronic endoscope 100 or the like, and the operating mode is switched according to a user operation performed on the mode switching button.

Also, although an example in which the present invention is applied to an electronic endoscope apparatus is described in the above embodiment, the present invention is not limited to this configuration. For example, the present invention can be applied to a video playback apparatus (or a video playback program for a personal computer) for playing back an endoscopic video captured by an electronic endoscope apparatus.

Moreover, the present invention is also applicable to the analysis of observed images other than endoscopic images (e.g., observed images of a body surface or observed images of the interior of a body during surgery, which have been captured by a normal video camera or still camera).

1-12. (canceled)

13. An image processing apparatus comprising:
an image data acquiring unit configured to acquire image data expressing a captured image of biological tissue; and
a YC separation processing unit configured to perform signal processing in which a luminance signal and a color signal are generated based on an RGB signal of the image data,
wherein a ratio of an R component of the RGB signal included in the luminance signal is greater than a ratio of a G component and a ratio of a B component.

14. The image processing apparatus according to claim 13,
wherein the signal processing includes
standard signal processing in which the image does not change substantially before and after the signal processing, and
special signal processing in which the luminance signal that is output includes a larger amount of the R component than both the G component and the B component,
the image processing apparatus comprises:
a selecting unit configured to select whether the standard signal processing or the special signal processing is to be performed, and
the YC separation processing unit performs the signal processing that was selected by the selecting unit.

15. The image processing apparatus according to claim 14,
wherein the YC separation processing unit performs the signal processing with use of a matrix operation that uses a color matrix, and
the YC separation processing unit uses a standard color matrix in the standard signal processing, and uses a special color matrix in the special signal processing.

16. The image processing apparatus according to claim 15,
wherein the YC separation processing unit includes:
a memory that stores the standard color matrix and the special color matrix;
a matrix selection unit that selects one of the standard color matrix and the special color matrix and reads out the selected matrix from the memory; and
a computation unit that performs the matrix operation with use of the matrix that was read out by the matrix selection unit.

17. The image processing apparatus according to claim 13, wherein the luminance signal is proportional to the R component of the RGB signal.

18. The image processing apparatus according to claim 13,
wherein the luminance signal includes an element obtained by multiplying the R component of the RGB signal by a gain constant, and
the image processing apparatus comprises:
a unit configured to change the gain constant.

19. The image processing apparatus according to claim 18, comprising:
an automatic gain adjusting unit configured to automatically adjust the gain constant based on the luminance signal.

20. The image processing apparatus according to claim 13, wherein the ratio of the R component of the RGB signal included in the luminance signal is greater than a sum of the ratio of the G component and the ratio of the B component.

21. The image processing apparatus according to claim 13, wherein the ratio of the R component of the RGB signal included in the luminance signal is greater than or equal to 50%.

22. The image processing apparatus according to claim 13, wherein the color signal is made up of two color difference signals.

23. The image processing apparatus according to claim 13, wherein the YC separation processing unit generates a YCrCb signal, a YPrPb, or a YUV signal.

24. An image processing apparatus comprising:
an image data acquiring unit configured to acquire image data expressing a captured image of biological tissue; and
a YC separation processing unit configured to perform signal processing in which a luminance signal and a color signal are generated based on an RGB signal of the image data,
wherein the signal processing includes
standard signal processing in which the image does not change substantially before and after the signal processing, and
special signal processing in which the luminance signal that is output includes a larger amount of the R component of the RGB signal than in the standard signal processing,
the image processing apparatus comprises:
a selecting unit configured to select whether the standard signal processing or the special signal processing is to be performed, and
the YC separation processing unit performs the signal processing that was selected by the selecting unit.
